(19) United States
(12) Patent Application Publication
(10) Pub. No.: US 2024/0024210 A1
(43) Pub. Date: Jan. 25, 2024

(54) MINERAL-OIL-FREE W/O EMULSION WITH A VARIABLE CONSISTENCY

(71) Applicant: BEIERSDORF AG, Hamburg (DE)

(72) Inventors: Jessica RATHSACK, Buxtehude (DE); Tanja HARTWIG, Bad Oldeslohe (DE); Dorothe FIEDLER, Hamburg (DE)

(21) Appl. No.: 17/999,244

(22) PCT Filed: Apr. 30, 2021
(86) PCT No.: PCT/EP2021/061399

§ 371 Date: May 25, 2023

(30) Foreign Application Priority Data
May 27, 2020 (DE) 10 2020 003 169.2

(51) Int. Cl. A61K 8/06; A61K 8/37; A61K 8/92; A61K 8/34; A61K 8/02; A61Q 19/00

(52) U.S. Cl. CPC A61K 8/064; A61K 8/375; A61K 8/922; A61K 8/37; A61K 8/925; A61K 8/345; A61K 8/0229; A61Q 19/001

(57) ABSTRACT

Cosmetic water-in-oil emulsion (W/O emulsion) containing a first emulsifier from the group of the compounds diisostearoyl polyglyceryl-3 dimer dilinoleate and/or polyglyceryl-3 polyricinoleate, a second emulsifier from the group of the compounds polyglyceryl-4 diisostearate/polyhydroxystearate/sebacate and/or polyglyceryl-2 sesquioleate as well as sunflower wax, wherein the preparation is free from mineral oil, paraffin wax, microcrystalline wax, shellac wax and polyethylene wax, free from polyacrylates, Acrylates/C10-30 Alkyl Acrylate Crosspolymers and vinylpyrrolidone/hexadecene copolymers, as well as free from 3-(4-methylbenzylidene)-camphor, 2-hydroxy-4-methoxybenzophenone (INCI: Oxybenzone), 4-methoxycinnamic acid-2-ethylhexyl ester (INCI: Octyl Methoxycinnamate), ethylhexyl-2-cyano-3,3-di-diphenylacrylate (INCI: Octocrylene), parabens (in particular methyl-, propyl- and butylparaben), methylisothiazolinone, chloromethylisothiazolinone and DMDM hydantoin, polyethylene glycol ethers or polyethylene glycol esters, and production method.

The present invention relates to a cosmetic water-in-oil emulsion (W/O emulsion) comprising a first emulsifier from the group of the compounds diisostearoyl polyglyceryl-3 dimer dilinoleate and/or polyglyceryl-3 polyricinoleate, a second emulsifier from the group of the compounds polyglyceryl-4 diisostearate/polyhydroxystearate/sebacate and/or polyglyceryl-2 sesquioleate as well as sunflower wax, wherein the preparation is free from mineral oil, paraffin wax, microcrystalline wax, shellac wax and polyethylene waxes, free from polyacrylates, acrylate/C10-C30 alkyl acrylate crosspolymers and vinylpyrrolidone/hexadecene copolymers, and free from 3-(4-methylbenzylidene)camphor, 2-hydroxy-4-methoxybenzophenone (INCI: Oxybenzone), 2-ethylhexyl 4-methoxycinnamate (INCI Octyl Methoxycinnamate), ethylhexyl 2-cyano-3,3-diphenylacrylate (INCI: Octocrylene, parabens (particularly methyl, propyl and butyl paraben), methylisothiazolinone, chloromethylisothiazolinone and DMDM hydantoin, polyethylene glycol ethers or polyethylene glycol esters, and preparation processes thereof.

The preparation can be used in particular as a lip care preparation (lipstick, lip cream).

The desire to look beautiful and attractive has been ingrained in humans for thousands of years. Although ideals of beauty have changed over time, the pursuit of a flawless appearance has always been aimed for by humans, since a likeable appearance increases their self-esteem and attraction to those around them.

The term decorative cosmetics derives from the Latin “decoratio”—emphasizing beauty. In most cases, individual parts of the body, especially the face, are emphasized with the help of colorants and color inconsistencies are softened.

In addition to face powders and rouge as powdery cosmetics and stick-like preparations, creamy preparations such as day creams and emulsion-based cream make-up are used for this purpose.

Lipstick is one of the most used decorative cosmetics. More than 50% of women in Germany use it. In addition to accentuating the color of the lips, it is usually also used for lip care.

Lipsticks generally consist of a wax matrix into which liquid and semi-solid oils and also pigments and fillers are incorporated at low concentrations. They are in solid stick form.

A disadvantage of the prior art is the fact that lip care products such as lip creams, lip butter, etc., and in particular the lipsticks of the prior art, often comprise mineral oils and paraffin waxes, which are increasingly unpopular with consumers. In addition to a general desire for products made from renewable raw materials, concern about impurities hazardous to health that may be present in crude oil-based raw materials also plays a role. Whether this concern is actually justified can remain open in the context of the present disclosure. The fact is that there is a desire among many consumers for “mineral oil-free” products. It was therefore the object of the present invention to develop a “mineral oil-free” lip care product.

For the production of lipsticks in particular, however, dispensing with mineral oils and paraffin waxes has serious disadvantages. During production, the cooling process of the molten stick mass easily leads to visual defects, cracks and fractures in the stick mass.

It was therefore the object of the present invention to avoid the disadvantages of the prior art and to develop a lipstick that is free from mineral oil products and can be produced in high quality.

In particular, mineral oil-free lip care products (especially lipsticks) of the prior art also have the disadvantage that they are not particularly storage-stable, but are relatively sensitive to temperature and light, i.e., they become inhomogeneous and brittle/cracked relatively easily with temperature fluctuations or light. In addition, sticks of the prior art that are free of mineral oil have the disadvantage that, compared to sticks containing mineral oil, they exhibit poorer gliding properties on the lip. These sticks feel waxy/sticky/unpleasant.

It was therefore the object of the present invention to also reduce these deficiencies of the prior art.

Last but not least, a fundamental problem in the development of lip care products is that separate formulas with different compositions have to be developed for each preparation form (stick, cream, etc.). This does not only mean additional effort in the development and manufacture of the products. In addition, users have the problem that if they are very satisfied with a product, for example a lipstick, when they change product form to, for example, a cream, they cannot find a product with the same performance spectrum (for example in terms of tolerance, skin moisturization, etc.) because lipsticks and creams have different compositions in terms of their formulation.

It was therefore the object of the present invention to develop a cosmetic care formulation that can be processed into (physically) different product types (for example stick and cream).

Surprisingly, the objects are achieved by a cosmetic water-in-oil emulsion (W/O emulsion) containing
a) a first emulsifier from the group of the compounds diisostearoyl polyglyceryl-3 dimer dilinoleate and/or polyglyceryl-3 polyricinoleate
b) a second emulsifier from the group of the compounds polyglyceryl-4 diisostearate/polyhydroxystearate/sebacate and/or polyglyceryl-2 sesquioleate
c) sunflower wax, wherein the preparation is free from mineral oil, paraffin wax, microcrystalline wax, shellac wax and polyethylene waxes, free from polyacrylates, acrylate/C10-C30 alkyl acrylate crosspolymers and vinylpyrrolidone/hexadecene copolymers, and free from 3-(4-methylbenzylidene)camphor, 2-hydroxy-4-methoxybenzophenone (INCI: Oxybenzone), 2-ethylhexyl 4-methoxycinnamate (INCI Octyl Methoxycinnamate), ethylhexyl 2-cyano-3,3-diphenylacrylate (INCI: Octocrylene, parabens (particularly methyl, propyl and butyl paraben), methylisothiazolinone, chloromethylisothiazolinone and DMDM hydantoin, polyethylene glycol ethers or polyethylene glycol esters.

Surprisingly, the objects are further achieved by a process for producing a cosmetic water-in-oil emulsion (W/O emulsion), wherein the oil phase contains
a) a first emulsifier from the group of the compounds diisostearoyl polyglyceryl-3 dimer dilinoleate and/or polyglyceryl-3 polyricinoleate
b) a second emulsifier from the group of the compounds polyglyceryl-4 diisostearate/polyhydroxystearate/sebacate and/or polyglyceryl-2 sesquioleate and
c) sunflower wax, wherein the preparation is free from mineral oil, paraffin wax, microcrystalline wax, shellac wax and polyethylene waxes, free from polyacrylates, acrylate/C10-C30 alkyl acrylate crosspolymers and vinylpyrrolidone/hexadecene copolymers, and free from 3-(4-methylbenzylidene)camphor, 2-hydroxy-4-methoxybenzophenone (INCI: Oxybenzone), 2-ethylhexyl 4-methoxycinnamate (INCI Octyl Methoxycinnamate), ethylhexyl 2-cyano-3,3-diphenylacrylate (INCI: Octocrylene, parabens (in particular methyl, propyl and butyl paraben), methylisothiazolinone, chloromethylisothiazolinone and DMDM hydantoin, polyethylene glycol ethers or polyethylene glycol esters, characterized in that the oil phase, the aqueous phase and the oil phase are first combined and homogenized at a temperature equal to/greater than 80° C. in each case.

In the context of the present disclosure, the phrases “according to the invention”, “preparation according to the invention”, etc. refer in all cases to the preparations and processes according to the invention, i.e., also to preparations with which the process according to the invention is executed.

It is advantageous in the context of the present invention if diisostearoyl polyglyceryl-3 dimer dilinoleate is used as first emulsifier and polyglyceryl-4 diisostearate/polyhydroxystearate/sebacate is used as second emulsifier.

The preparations according to the invention are advantageously characterized in that the total concentration of the (one) or of the (two) first emulsifier(s) is 1 to 4% by weight, based on the total weight of the preparation. In this case, preference is given in accordance with the invention to a total concentration of 1.2 to 2.5% by weight, based on the total weight of the preparation.

Furthermore, the advantageous embodiments of the present invention according to the invention are characterized in that the total concentration of the second emulsifier(s) is 0.5 to 2.5% by weight, based on the total weight of the preparation. In this case, preference is given in accordance with the invention to a total concentration of 0.8 to 1.5% by weight, based on the total weight of the preparation.

The use concentration for sunflower wax which is advantageous in accordance with the invention is 1 to 10% by weight, based on the total weight of the preparation. In this case, a use concentration of 1.5 to 4.5% by weight, based on the total weight of the preparation, is preferred in accordance with the invention.

It is also advantageous in the context of the present invention if the amount of the water phase is 35 to 65% by weight and the amount of the oil phase is 35 to 65% by weight, based on the total amount of the preparation. The sum total of both phases should advantageously be 100% by weight.

Embodiments of the present invention that are advantageous according to the invention are also obtained in that the preparation contains esters, of which the alcohol and acid unit of each comprises hydrocarbon chains of chain length C14 to C62.

It is preferred according to the invention if the preparation comprises behenyl behenate (INCI: Behenyl Behenate).

Furthermore, it is advantageous according to the invention if the preparation according to the invention comprises beeswax (INCI: Cera alba). The use concentration for beeswax that is advantageous in accordance with the invention is 1 to 10% by weight, based on the total weight of the preparation.

It was particularly surprising for the person skilled in the art that no additional salts are required to stabilize the W/O emulsion, as is usually the case for W/O emulsions. Rather, it is advantageous in the context of the present invention if the salt content of the preparation is less than/equal to 0.1% by weight, based on the total weight of the preparation.

In addition, it is advantageous in the context of the present invention if the preparation comprises glycerin. In such a case, the preferred embodiments of the present invention according to the invention are characterized in that the preparation comprises glycerin in an amount of 1 to 20% by weight, based on the total weight of the preparation.

In addition, it is advantageous in accordance with the invention if the preparation comprises one or more compounds selected from the group of the compounds alpha-lipoic acid, folic acid, phytoene, D-biotin, coenzyme Q10, alpha-glucosylrutin, carnitine, carnosine, natural and/or synthetic isoflavonoids, flavonoids, creatine, creatinine, taurine, β-alanine, panthenol, magnolol, honokiol, tocopheryl acetate, dihydroxyacetone; thiamidol, 8-hexadecene-1,16-dicarboxylic acid, glycerylglucose, allantoin, vitamin E or vitamin E acetate, hyaluronic acid and/or salts thereof, licochalcone A and/or plant extracts.

Preferred according to the invention in this case is the use of the compounds panthenol, allantoin and hyaluronic acid.

Furthermore, the emulsion according to the invention may comprise the corresponding, customary cosmetic ingredients and lipophilic components, according to its intended use.

One of the special features of the present invention that could not be foreseen by a person skilled in the art is the fact that either flowable creams and ointments or solid stick formulations (for example lipsticks) can be produced with one and the same recipe, depending on the production process. Once the emulsion according to the invention has been produced by means of the hot-hot process, it can be further processed into a cream/lotion or a stick mass according to the further process steps. It is also possible to transfer creams/lotions/ointments or sticks already produced to the relevant other product category by simply heating them while applying the further process steps, resulting in new, resource-saving options in the processing of production surpluses.

To produce a cream, lotion or ointment, the procedure is as follows:

Here, the process according to the invention is characterized in that after the homogenization at a temperature of greater than/equal to 80° C., the preparation is cooled to 40 to 45° C. while stirring and is then homogenized again.

In a further process step which is advantageous according to the invention, the preparation according to the invention is further cooled after the second homogenization and filled into storage containers at a temperature of 35 to 40° C.

To produce a stick-shaped formulation, on the other hand, the procedure is as follows:

Here, the process according to the invention is characterized in that after the homogenization at a temperature of greater than/equal to 80° C., the preparation is cooled to 70 to 75° C. while stirring and then poured into a casting mold for sticks and cooled therein to room temperature at a cooling rate of 0.2-0.4° C./sec.

The cooling rate after casting the stick should therefore be as high as possible.

A stick produced in this way still has sufficient dimensional stability even at 40° C., so that application is possible without the stick breaking off or being crushed.

According to the invention, this method for producing sticks is advantageously characterized in that, after cooling to room temperature, the stick-shaped mass (stick pencil) is removed from the mold and transferred to a storage container for stick-shaped preparations.

### EXAMPLES

The examples below are intended to illustrate the present invention without limiting it. Unless otherwise stated, all quantitative data, fractions, and percentages are based on the weight and the total amount or on the total weight of the preparations.

| | | Example | Example | Example | Example |
|---|---|---|---|---|---|
| | INCI | 1 | 2 | 3 | 4 |
| Phase | Behenyl Behenate | 5 | 4 | | 4 |
| A | Prunus Amygdalus Dulcis Oil | 20 | 20 | 20 | 20 |
| | Cera Alba | | | 4 | |
| | Ricinus Communis Seed Oil | 10 | 10 | 10 | 10 |
| | Helianthus Annuus Seed Wax | 1 | 2.5 | 2 | 2.5 |
| | Diisostearoyl Polyglyceryl-3 Dimer Dilinoleate | 1.4 | 1.4 | 1.4 | |
| | Polyglyceryl-4 | 0.8 | 0.8 | 0.8 | |
| | Diisostearate/Polyhydroxystearate/Sebacate | | | | |
| | Polyglyceryl-3 Polyricinoleate | | | | 1.4 |
| | Polyglyceryl-2 Sesquioleate | | | | 0.8 |
| Phase | Phenoxyethanol | 0.8 | 0.8 | 0.8 | 0.8 |
| B | Glyceryl Caprate | 0.3 | 0.3 | 0.3 | 0.3 |
| | Glycerin | 10 | 10 | 10 | 10 |
| | Caprylyl Glycol | 0.15 | 0.15 | 0.15 | 0.15 |
| | Aqua | 50.55 | 50.05 | 50.55 | 50.05 |
| | | 100 | 100 | 100 | 100 |

### Production of Solid Formulation

Heat phase A to 90° C. while stirring
Heat phase B to 85° C. while stirring
Slowly add phase B to phase A, stirring at 800-1200 rpm
Stir the emulsion at 80° C. for 20 minutes
Then pour into the mold at 70-75° C.
Leave to cool at room temperature for 15 minutes. The cooling rate here is ca. 0.3° C./second.

Then allow to harden for a further 30 minutes at −18° C.
Recommended equipment: IKA agitator with suitable dissolver

### Production of Cream Formulation

Heat phase A to 90° C. while stirring
Heat phase B to 85° C. while stirring
Slowly add phase B to phase A, stirring at 150 rpm
Homogenize hot emulsion
Then stir cold (50-70 rpm)
Homogenize the emulsion again at 30° C.

1. Cosmetic water-in-oil emulsion (W/O emulsion) containing
a) a first emulsifier from the group of the compounds diisostearoyl polyglyceryl-3 dimer dilinoleate and/or polyglyceryl-3 polyricinoleate
b) a second emulsifier from the group of the compounds polyglyceryl-4 diisostearate/polyhydroxystearate/sebacate and/or polyglyceryl-2 sesquioleate
c) sunflower wax, wherein the preparation is free from mineral oil, paraffin wax, microcrystalline wax, shellac wax and polyethylene waxes, free from polyacrylates, acrylate/C10-C30 alkyl acrylate crosspolymers and vinylpyrrolidone/hexadecene copolymers, and free from 3-(4-methylbenzylidene)camphor, 2-hydroxy-4-methoxybenzophenone (INCI: Oxybenzone), 2-ethylhexyl 4-methoxycinnamate (INCI Octyl Methoxycinnamate), ethylhexyl 2-cyano-3,3-diphenylacrylate (INCI: Octocrylene, parabens (particularly methyl, propyl and butyl paraben), methylisothiazolinone, chloromethylisothiazolinone and DMDM hydantoin, polyethylene glycol ethers or polyethylene glycol esters.

2. Process for producing a cosmetic water-in-oil emulsion (W/O emulsion), wherein the oil phase contains
a) a first emulsifier from the group of the compounds diisostearoyl polyglyceryl-3 dimer dilinoleate and/or polyglyceryl-3 polyricinoleate
b) a second emulsifier from the group of the compounds polyglyceryl-4 diisostearate/polyhydroxystearate/sebacate and/or polyglyceryl-2 sesquioleate and
c) sunflower wax, wherein the preparation is free from mineral oil, paraffin wax, microcrystalline wax, shellac wax and polyethylene waxes, free from polyacrylates, acrylate/C10-C30 alkyl acrylate crosspolymers and vinylpyrrolidone/hexadecene copolymers, and free from 3-(4-methylbenzylidene)camphor, 2-hydroxy-4-methoxybenzophenone (INCI: Oxybenzone), 2-ethylhexyl 4-methoxycinnamate (INCI Octyl Methoxycinnamate), ethylhexyl 2-cyano-3,3-diphenylacrylate (INCI: Octocrylene, parabens (in particular methyl, propyl and butyl paraben), methylisothiazolinone, chloromethylisothiazolinone and DMDM hydantoin, polyethylene glycol ethers or polyethylene glycol esters, characterized in that the oil phase, the aqueous phase and the oil phase are first combined and homogenized at a temperature equal to/greater than 80° C. in each case.

3. W/O emulsion according to claim 1 or process according to claim 2, characterized in that diisostearoyl polyglyceryl-3 dimer dilinoleate is used as first emulsifier and polyglyceryl-4 diisostearate/polyhydroxystearate/sebacate is used as second emulsifier.

4. W/O emulsion or process according to one of the preceding claims, characterized in that the total concentration of the first emulsifier(s) is 1 to 4% by weight, based on the total weight of the preparation.

5. W/O emulsion or process according to one of the preceding claims, characterized in that the total concentration of the second emulsifier(s) is 0.5 to 2.5% by weight, based on the total weight of the preparation.

6. W/O emulsion or process according to one of the preceding claims, characterized in that the concentration of sunflower wax is 1 to 10% by weight, based on the total weight of the preparation.

7. W/O emulsion or process according to one of the preceding claims, characterized in that the amount of the water phase is 35 to 65% by weight and the amount of the oil phase is 35 to 65% by weight, based on the total amount of the preparation.

8. W/O emulsion or process according to one of the preceding claims, characterized in that the preparation comprises esters, of which the alcohol and acid unit each comprises hydrocarbon chains of chain length C14 to C62.

9. W/O emulsion or process according to one of the preceding claims, characterized in that the preparation contains behenyl behenate (INCI: Behenyl Behenate).

10. W/O emulsion or process according to one of the preceding claims, characterized in that the preparation contains beeswax (INCI: Cera Alba).

11. W/O emulsion or process according to one of the preceding claims, characterized in that the salt content of the preparation is less than/equal to 0.1% by weight, based on the total weight of the preparation.

12. W/O emulsion or process according to one of the preceding claims, characterized in that the preparation comprises glycerin in an amount of 1 to 20% by weight, based on the total weight of the preparation.

13. W/O emulsion or process according to one of the preceding claims, characterized in that the preparation comprises one or more compounds selected from the group of the compounds alpha-lipoic acid, folic acid, phytoene, D-biotin, coenzyme Q10, alpha-glucosylrutin, carnitine, carnosine, natural and/or synthetic isoflavonoids, flavonoids, creatine, creatinine, taurine, β-alanine, panthenol, magnolol, honokiol, tocopheryl acetate, dihydroxyacetone; thiamidol, 8-hexadecene-1,16-dicarboxylic acid, glycerylglucose, allantoin, vitamin E and vitamin E acetate, hyaluronic acid and/or salts thereof, licochalcone A and/or plant extracts.

14. Process according to one of the preceding claims, characterized in that after the homogenization at a temperature of greater than/equal to 80° C., the preparation is cooled to 40 to 45° C. while stirring and is then homogenized again.

15. Process according to claim 14, characterized in that the preparation is cooled further after the second homogenization and is filled into storage containers at a temperature of 35 to 40° C.

16. Process according to one of claims 1 to 13, characterized in that after the homogenization at a temperature of greater than/equal to 80° C., the preparation is cooled to 70 to 75° C. while stirring and then poured into a casting mold for sticks and cooled therein to room temperature at a cooling rate of 0.2-0.4° C./second.

17. Process according to claim 16, characterized in that after cooling to room temperature the stick-shaped mass (stick pencil) is removed from the mold and transferred to a storage container for stick-shaped preparations.
